(19) United States
(12) Patent Application Publication
(10) Pub. No.: US 2023/0284984 A1
(43) Pub. Date: Sep. 14, 2023

(54) PET APPARATUS, PET-CT APPARATUS, IMAGE GENERATION AND DISPLAY METHOD, AND NONVOLATILE COMPUTER-READABLE STORAGE MEDIUM STORING IMAGE GENERATION AND DISPLAY PROGRAM

(71) Applicant: CANON MEDICAL SYSTEMS CORPORATION, Otawara-shi (JP)

(72) Inventor: Kanji Watanabe, Nasushiobara (JP)

(73) Assignee: CANON MEDICAL SYSTEMS CORPORATION, Otawara-shi (JP)

(21) Appl. No.: 18/164,850

(22) Filed: Feb. 6, 2023

(30) Foreign Application Priority Data
Mar. 8, 2022 (JP) 2022-035370

(51) Int. Cl. A61B 6/03; A61B 6/00

(52) U.S. Cl. CPC A61B 6/037; A61B 6/4266; A61B 6/4258

(57) ABSTRACT

A positron emission tomography (PET) apparatus includes a plurality of PET detector rings and processing circuitry. The PET detector rings are movable relative to a table top on which a subject is to be laid, in an axial direction of a bore into which the table top is to be inserted. The processing circuitry is configured to obtain position information of each of the plurality of PET detector rings in the axial direction, generate display information representing positions of the plurality of PET detector rings, based on the position information, and display the display information on a display.

### CROSS-REFERENCE TO RELATED APPLICATION

This application is based upon and claims the benefit of priority from Japanese Patent Application No. 2022-035370, filed on Mar. 8, 2022; the entire contents of which are incorporated herein by reference.

### FIELD

Embodiments described herein relate generally to a PET apparatus, a PET-CT apparatus, an image generation and display method, and a nonvolatile computer-readable storage medium storing an image generation and display program.

### BACKGROUND

Traditionally, positron emission tomography (PET) apparatuses include a PET ring in which PET detectors to detect photons are arranged in a ring form. In recent years, PET apparatuses with multiple PET rings have been developed. Multiple PET rings may be configured to be freely movable in a body axis direction. Freely movable PET rings can be appropriately changed in position in accordance with a scanning range length along the body axis of a subject.

In the case of scanning the whole body of the subject or a particular region of the subject, however, the user may face difficulty with knowing the positions of the individual PET rings while moving the PET rings in accordance with the scanning range. For this reason, it may be difficult to efficiently move the PET rings to place the PET rings at some positions.

### BRIEF DESCRIPTION OF THE DRAWINGS

FIG. 1 illustrates an exemplary structure of a PET-CT apparatus according to a first embodiment;

FIG. 2 illustrates examples of arrangement of multiple PET detector rings according to a scanning range in the first embodiment;

FIG. 3 is a flowchart illustrating an information generation/display process of the first embodiment by way of example;

FIG. 4 illustrates exemplary display information displayed on a display in the first embodiment;

FIG. 5 illustrates exemplary display information representing that a localizer image of a subject is being swiped from the head to the legs at step S306 in the first embodiment;

FIG. 6 illustrates exemplary display information representing that a localizer image of a subject is being swiped from the shoulder to the lumber region at step S306 in the first embodiment;

FIG. 7 illustrates an exemplary user operation with respect to the display information illustrated in FIG. 4 in the first embodiment;

FIG. 8 illustrates an exemplary count-rate map displayed on a display according to a first application of the first embodiment;

FIG. 9 illustrates differences in arrangement of multiple PET detector rings with respect to a subject between a first scan and a second scan according to a fourth application of the first embodiment;

FIG. 10 illustrates projection positions of laser emitted from projectors in a modification of the first embodiment by way of example;

FIG. 11 illustrates an imaging area of a first camera and an imaging area of a second camera in a modification of the first embodiment;

FIG. 12 is a flow diagram illustrating an exemplary operation procedure of multiple PET detector rings in a PET-CT examination in a second embodiment; and

FIG. 13 illustrates positions of multiple PET detector rings evacuated outside an X-ray irradiation range according to the second embodiment.

### DETAILED DESCRIPTION

According to an embodiment, a positron emission tomography (PET) apparatus includes a plurality of PET detector rings and processing circuitry. The PET detector rings are movable relative to a table top on which a subject is to be laid, in an axial direction of a bore into which the table top is to be inserted. The processing circuitry is configured to obtain position information of each of the plurality of PET detector rings in the axial direction, generate display information representing positions of the plurality of PET detector rings, based on the position information, and display the display information on a display.

Hereinafter, embodiments of a positron emission tomography (PET) apparatus, a PET-computed tomography (CT) apparatus, an image generation/display method, and an image generation/display program will be described in detail with reference to the accompanying drawings. In various embodiments to be described below, parts, portions, elements, or functions denoted by the same reference numerals are considered to perform same or similar operation, and an overlapping explanation thereof will be omitted when appropriate. The following embodiments are presented for illustrative purpose only and not intended to limit the features of a PET apparatus, a PET-CT apparatus, an image generation/display method, and an image generation/display program of this disclosure.

The PET apparatus of some embodiments includes an imaging system that performs PET imaging (PET scan and PET localizer scan). Examples of such a PET apparatus include a PET apparatus with a PET imaging function only, a PET-CT apparatus including a PET imaging system and an X-ray computed tomography (CT) imaging system, a PET-magnetic resonance (MR) apparatus including a PET imaging system and an MR imaging system, and so on. The following embodiments will describe a PET-CT apparatus as an example for the sake of concreteness. The PET imaging system of some embodiments includes multiple PET detector rings.

### First Embodiment

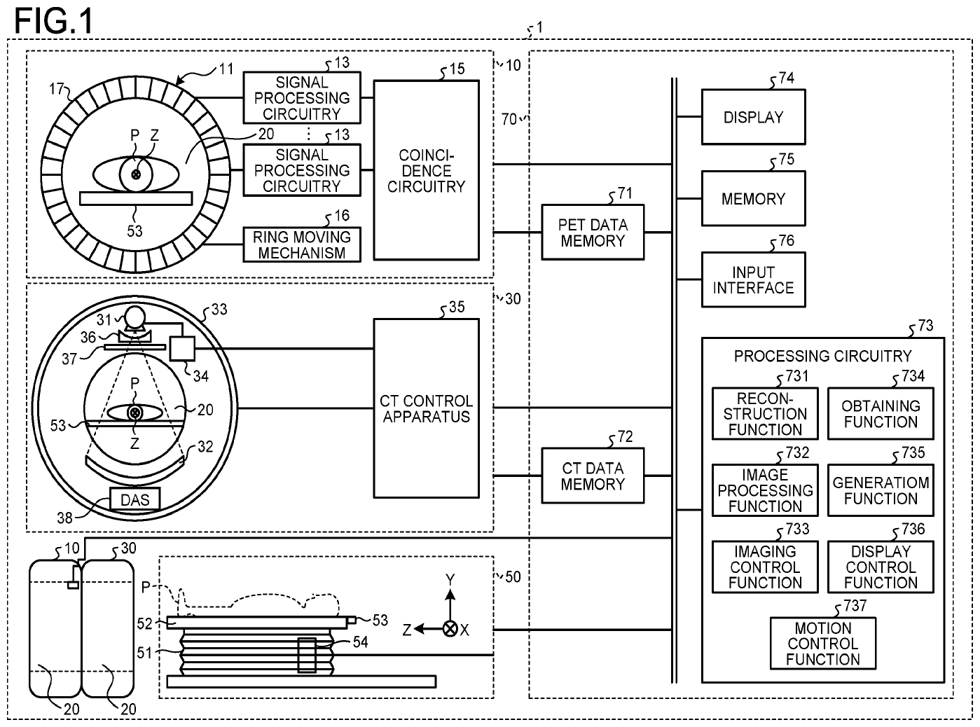

FIG. 1 illustrates an exemplary structure of a PET-CT apparatus 1 according to a first embodiment. As illustrated in FIG. 1, the PET-CT apparatus 1 includes a PET gantry 10, a CT gantry 30, a couch 50, and a console 70. Typically, the PET gantry 10, the CT gantry 30, and the couch 50 are installed together in the same examination room while the console 70 is installed in a control room adjacent to the examination room. The PET gantry 10 is an imaging apparatus for PET imaging (i.e., PET scan and/or PET localizer scan) of a subject P. The CT gantry 30 is an imaging apparatus for CT imaging (i.e., CT scan and/or CT localizer scan) of the subject P with X-rays. The couch 50 movably supports a table top 53 on which the subject P to be scanned is laid. The console 70 is a computer that performs control over the PET gantry 10, the CT gantry 30, and the couch 50.

The PET gantry 10 includes, for example, multiple PET detector rings, signal processing circuitry 13, coincidence circuitry 15, and a ring moving mechanism 16. Although FIG. 1 depicts a single PET detector ring 11 alone, an actual PET gantry 10 incorporates two or more PET detector rings which are movable relative to the table top 53 on which the subject P is to be laid, in an axial direction (Z-direction) of a bore 20 into which the table top 53 is to be inserted. The signal processing circuitry 13 and the coincidence circuitry 15 are, for example, provided for each of the PET detector rings. The ring moving mechanism 16 movably supports the individual PET detector rings in the axial direction (Z-direction) of the bore 20. The PET gantry 10 and the CT gantry 30 may be contained in the same casing.

Each PET detector ring 11 includes multiple gamma-ray detectors 17 arranged in the circumference around the axis Z. The gamma-ray detectors 17 are also referred to as PET detectors. The opening of the PET detector ring 11 has an image field of view (FOV) set. The subject P is localized so as to set a region of interest of the subject P in the image FOV. The subject P is given a medical agent labelled with positron-emitting radionuclides. Positrons emitted from the positron-emitting radionuclides and the surrounding electrons annihilate each other. By annihilation, a pair of annihilation gamma rays is generated. The gamma-ray detectors 17 detect annihilation gamma rays emitted from the internal body of the subject P. The gamma-ray detectors 17 generate an electric signal in accordance with an amount of the detected annihilation gamma rays. For example, the gamma-ray detectors 17 each include multiple scintillators and multiple photoelectron multipliers. The scintillators receive annihilation gamma rays derived from the radioactive isotopes inside the subject P to generate scintillation light. The photoelectron multipliers generate electric signals in accordance with an amount of the scintillation light and supply the electric signals to the signal processing circuitry 13.

The signal processing circuitry 13 serves to generate single-event data from the electric signals output from the gamma-ray detectors 17. Specifically, the signal processing circuitry 13 subjects the electric signals to, for example, detection time measurement, position computation, and energy computation. The signal processing circuitry 13 can be implemented by, for example, an application specific integrated circuit (ASIC), a field programmable gate array (FPGA), a complex programmable logic device (CPLD), or a simple programmable logic device (SPLD) configured to be able to perform detection time measurement, position computation, and energy computation.

In the detection time measurement process, the signal processing circuitry 13 measures the time at which the gamma-ray detectors 17 detect the gamma rays. Specifically, the signal processing circuitry 13 monitors the crest value of the electric signals from the gamma-ray detectors 17 to measure the time at which the crest value exceeds a preset threshold. Such time is defined as a detection time. In other words, the signal processing circuitry 13 electrically detects annihilation gamma rays by detecting the crest value's exceeding the threshold. In the position computation process, the signal processing circuitry 13 computes the incident positions of the annihilation gamma rays in accordance with the electric signals from the gamma-ray detectors 17. The incident positions of the annihilation gamma rays correspond to the positional coordinates of the scintillators on which the annihilation gamma rays are incident. In the energy computation process, the signal processing circuitry 13 computes the energy value of the detected annihilation gamma rays in accordance with the electric signals from the gamma-ray detectors 17.

With respect to a single event, detection time data, positional coordinate data, and energy-value data are associated with one another. A combination of energy-value data, position coordinate data, and detection time data about a single event are referred to as single-event data. The signal processing circuitry 13 generates single-event data one after another upon detection of the annihilation gamma rays, and supplies the single-event data to the coincidence circuitry 15.

The coincidence circuitry 15 performs coincidence counting of the single-event data supplied from the signal processing circuitry 13. The coincidence circuitry 15 can be implemented by hardware resources such as an ASIC, an FPGA, a CPLD, or an SPLD configured to be able to perform coincidence counting. In the coincidence counting process, the coincidence circuitry 15 repeatedly identifies single-event data as to two single events occurring in a predefined time frame from among repeatedly supplied single-event data. It is inferred that such a single event pair is derived from the annihilation gamma rays generated at the same annihilate point. The single event pair is referred to as a coincidence event. The line connecting a pair of gamma-ray detectors 17 (specifically, scintillators) having detected the single event pair is referred to as a line of response (LOR). Event data on the event pair related to the LOR is referred to as coincidence event data. The coincidence event and the single-event data are transferred to the console 70. Hereinafter, the coincidence event data and the single-event data may be collectively referred to as PET event data unless the data items need to be distinguished.

The above has described an example that the signal processing circuitry 13 and the coincidence circuitry 15 are included in the PET gantry 10, however, the present embodiment is not limited to such an example. For example, the coincidence circuitry 15 or both the signal processing circuitry 13 and the coincidence circuitry 15 may be included in an apparatus independent from the PET gantry 10. Alternatively, a single piece of coincidence circuitry 15 may be provided for multiple pieces of signal processing circuitry 13 incorporated in the PET gantry 10. Multiple pieces of signal processing circuitry 13 incorporated in the PET gantry 10 may be classified into groups and a single piece of coincidence circuitry 15 may be provided for each of the groups.

The ring moving mechanism 16 works to move the multiple PET detector rings in the axial direction (Z-direction) of the bore 20 under the control of a motion control function 737 of processing circuitry 73 as described later. The ring moving mechanism 16 includes, for example, a ring support mechanism that movably supports the PET detector rings in the axial direction (Z-direction) of the bore 20, a moving mechanism that moves the PET detector rings in the ring support mechanism, and a drive mechanism that drives the moving mechanism. The ring support mechanism can be implemented by, for example, a linear motion bearing placed in the PET gantry 10 in the axial direction (Z-direction) of the bore 20. The ring support mechanism can be suitably implemented by any of various kinds of known bearings, in addition to the linear motion bearing. The rail for the linear motion bearing is set in a stationary frame of the PET gantry 10, extending in the axial direction (Z-direction) of the bore 20. In the linear motion bearing, the block which runs on the rail includes a support frame for maintaining the PET detector rings in a ring form.

The moving mechanism can be implemented by racks and pinions corresponding to the PET detector rings. In addition to the racks and pinions, the moving mechanism can be suitably implemented by any of known devices such as ball screws. Rack gears of the racks and pinions are arranged in the axial direction (Z-direction) of the bore 20 and individually connected to the support frames for the PET detector rings. Pinion gears are individually engaged with the rack gears. The pinion gears may be provided with, for example, a rotary encoder that measures the rotation rates of the pinion gears. In this case the output of the rotary encoder is output to the processing circuitry 73.

The drive mechanism can be implemented by, for example, a motor. The rotational shaft of the motor is, for example, connected to the pinion gears via various kinds of gears. Without the rotary encoder in the pinion gears, the rotational shaft of the motor or the various kinds of gears may be provided with, for example, a rotary encoder that measures the rotation rate of the rotational shaft. In this case the output of the rotary encoder is output to the processing circuitry 73. The motor is driven by a control signal from the motion control function 737. Along with the rotation of the motor, the pinion gears rotate to move the rack gears in the axial direction (Z-direction) of the bore 20. Along with the motion of the rack gears, the PET detector rings move in the axial direction (Z-direction) of the bore 20.

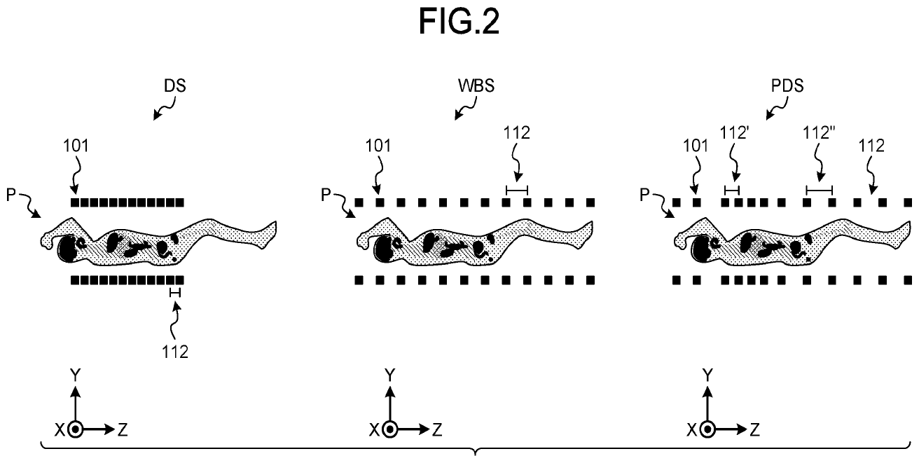

FIG. 2 illustrates examples of arrangement of PET detector rings 101 according to a scanning range. The left drawing of FIG. 2 shows an example that the PET detector rings 101 are densely arranged (hereinafter, dense state DS). In the dense state DS the PET detector rings 101 are arranged in line with, for example, a shorter or narrower scanning range of the subject P from a cervical region to a lumber region. In the dense state DS of FIG. 2, spacing 112 between every two adjacent PET detector rings is narrower than in the other drawings of FIG. 2. In such a state the PET gantry 10 can obtain data of a small region of interest of the subject P at a high signal-to-noise ratio (SNR).

The middle drawing of FIG. 2 shows an example that the PET detector rings 101 are uniformly arranged to cover the whole body of the subject P (hereinafter, whole-body state WBS). In the whole-body state WBS, the PET detector rings 101 are arranged in line with a longer or wider scanning range for the whole body of the subject P. In the whole-body state WBS of FIG. 2, the PET gantry 10 can obtain data of a large region of interest of the subject P in a longer scanning range. In such a state, spacing 112 between every two adjacent PET detector rings is wider than in the dense state DS of FIG. 2.

The right drawing of FIG. 2 shows an example that part of the PET detector rings 101 is arranged densely to cover a particular region of the subject P (hereinafter, partially dense state PDS). In the partially dense state PDS, the PET detector rings 101 as a whole are arranged to cover the whole body of the subject P in the scanning range and part of the PET detector rings 101 are arranged for the chest region of the subject P at a higher density than the rest. As illustrated in FIG. 2, the PET detector rings 101 according to the present embodiment are movable in the axial direction (Z-direction) of the bore 20, when appropriate.

The arrangement of the PET detector rings 101 is set, for example, depending on particular characteristics of the subject P including age, gender, and height of the subject P, radiation attenuation inside the subject P, an examination purpose, a user set scanning range, or else. A particular region of the subject P is set depending on a relevant factor of the subject P. For example, in the partially dense state PDS, spacing 112′ between two adjacent PET detector rings for PET imaging of a particular region corresponds to a first distance, and spacing 112″ between two adjacent PET detector rings for PET imaging of another particular region corresponds to a second distance longer than the first distance. As illustrated in the partially dense state PDS of FIG. 2, with respect to a region of interest of the subject P including the upper respiratory tract, the spacing 112′ between the PET detector rings for the upper torso is set shorter than the spacing 112″ between the PET detector rings for the lower limbs.

As illustrated in FIG. 1, the CT gantry 30 includes a CT imaging system. The CT imaging system performs a CT scan of the subject P. The CT imaging system may perform a localizer scan of the subject P with X-rays. The CT imaging system includes an X-ray tube 31, an X-ray detector 32, a rotational frame 33, an X-ray high voltage apparatus 34, a CT control apparatus 35, a wedge 36, a collimator 37, and a DAS 38.

The X-ray tube 31 generates X-rays. Specifically, the X-ray tube 31 includes a vacuum tube having a negative pole that generates thermoelectrons and a positive pole that generates X-rays by receiving the thermoelectrons flying from the negative pole. The X-ray tube 31 is connected to the X-ray high-voltage apparatus 34 via a high-voltage cable. The X-ray high voltage apparatus 34 applies a tube voltage between the positive pole and the negative pole. By applying the tube voltage, the thermoelectrons fly from the negative pole to the positive pole, which causes a tube current to flow. By a high-voltage application and a filament current supply from the X-ray high voltage apparatus 34, the thermoelectrons fly from the negative pole to the positive pole to collide with the positive pole. Thereby, X-rays are generated.

The X-ray detector 32 detects an X-ray emitted from the X-ray tube 31 and having passed through the subject P and outputs an electric signal corresponding to an amount of the X-ray to the DAS 38. The X-ray detector 32 has a structure that arrays of detection elements in the channel direction are arrayed in a slice direction (column or row direction), for example. The X-ray detector 32 is exemplified by an indirect-conversion detector including a grid, a scintillator array, and an optical sensor array. The scintillator array includes multiple scintillators and each scintillator outputs an amount of light corresponding to an amount of incident X-rays. The grid is disposed on the X-ray incident side of the scintillator array and includes an X-ray shield plate that functions to absorb scattered X-rays. The optical sensor array functions to convert the light from the scintillators into an electric signal corresponding to an amount of the light. Examples of the optical sensors include photo diodes and photoelectron multipliers. The X-ray detector 12 may be implemented by a direct-conversion detector (semiconductor detector) including a semiconductor element that converts an incident X-ray into an electric signal.

The rotational frame 33 is an annular frame that supports the X-ray tube 31 and the X-ray detector 32 rotatably about the rotational axis Z. Specifically, the rotational frame 33 supports the X-ray tube 31 and the X-ray detector 3 in opposing positions. The rotational frame 33 is supported by a stationary frame (not illustrated) rotatably about the rotational axis Z. The rotational frame 33 is rotated about the rotational axis Z under the control of the CT control apparatus 35. Thus, the X-ray tube 31 and the X-ray detector 32 are rotated about the rotational axis Z. The rotational frame 33 is supplied with power from the drive mechanism of the CT control apparatus 35 to rotate about the rotational axis Z at a constant angular rate. The opening of the rotational frame 33 has an image field of view (FOV) set.

In the present embodiment the rotational axis of the rotational frame 33 in a non-tilted state or a longitudinal direction of the table top 53 of the couch 50 is defined as a Z-axis direction. An axial direction orthogonal to the Z-axis direction and horizontal to the floor is defined as an X-axis direction. An axial direction orthogonal to the Z-axis direction and vertical to the floor is defined as a Y-axis direction.

The X-ray high voltage apparatus 34 includes electric circuitry such as a transformer and a rectifier. The high voltage generator further includes a high voltage generator and an X-ray control device. The high voltage generator generates a high voltage to be applied to the X-ray tube 31 and a filament current to be supplied to the X-ray tube 31. The X-ray control device controls the output voltage in accordance with the X-rays emitted from the X-ray tube 31. The high-voltage generator may be of a transformer type or of an inverter type. Further, the X-ray high-voltage apparatus 34 may be disposed in the rotational frame 33 or the stationary frame (not illustrated) of the CT gantry 30.

The wedge 36 serves to adjust the amount of X-rays to irradiate the subject P. Specifically, the wedge 36 attenuates the X-rays emitted from the X-ray tube 31 so that the subject P is irradiated with a dose of the X-rays in a predefined distribution. The wedge 36 is, for example, a metal plate such as aluminum. Examples of the wedge 36 include a wedge filter and a bow-tie filter.

The collimator 37 functions to limit the irradiation range of the X-rays having transmitted the wedge 36. The collimator 37 slidably supports lead plates forming slits and adjusts the form of the slits to shield the X-rays.

The data acquisition system (DAS) 38 functions to read electric signals from the X-ray detector 32. The electric signals correspond to the doses of X-rays detected by the X-ray detector 32. The DAS 38 amplifies the electric signals at a variable amplification rate and integrate the amplified electric signals in a view period to thereby acquire CT raw data having a digital value corresponding to the dose of X-rays during the view period. The DAS 38 can be implemented by, for example, an ASIC incorporating a circuit element capable of generating CT raw data. The CT raw data is transferred to the console 70 via a non-contact data transfer device.

The CT control apparatus 35 controls the X-ray high voltage apparatus 34 and the DAS 38 for performing a CT scan with X-rays under the control of an imaging control function 733 of the processing circuitry 73 in the console 70. The CT control apparatus 35 includes processing circuitry including a central processing unit (CPU) and a driving mechanism such as a motor and an actuator. The processing circuitry includes hardware resources such as a processor as a CPU or a micro-processing unit (MPU) and memory as read only memory (ROM) and random access memory (RAM). The CT control apparatus 35 may be implemented by an ASIC, an FPGA, a CPLD, or an SPLD.

Various types of the CT gantry 30 are available, including a rotate/rotate-type (third-generation CT) that the X-ray generator and the X-ray detector rotate about the subject in an integrated manner and a stationary/rotate-type (fourth-generation CT) including a large number of X-ray detector elements stationarily arrayed in a ring form that only the X-ray generator rotates about the subject. Any type of CT gantry is applicable to embodiments.

As illustrated in FIG. 1, the couch 50 is a device on which the subject P to be scanned is laid and moved. The couch 50 is shared with the PET gantry 10 and the CT gantry 30.

The couch 50 includes a base 51, a support frame 52, the table top 53, and a couch driver 54. The base 51 is a housing that supports the support frame 52 movably in a vertical (Y-axis) direction with respect to the floor. The support frame 52 is placed on the top of the base 51. The support frame 52 supports the table top 53 slidably along the axis Z. The table top 53 is a flexible plate on which the subject P is to be laid.

The couch driver 54 is accommodated in the housing of the couch 50. The couch driver 54 includes a motor or an actuator that generates power to move the support frame 52 and the table top 33 on which the subject P is laid. The couch driver 54 operates under the control of the console 70, for example.

The PET gantry 10 and the CT gantry 30 are disposed in a manner that the axis Z of the opening of the PET gantry 10 and the axis Z of the opening of the CT gantry 30 substantially match each other. The couch 50 is placed such that the long axis of the table top 53 becomes parallel to the axes Z of the openings of the PET gantry 10 and the CT gantry 30. The CT gantry 30 and the PET gantry 10 are, for example, disposed in this order relative to the couch 50. Namely, the CT gantry 30 is closer to the couch 50 than the PET gantry 10.

As illustrated in FIG. 1, the console 70 includes a PET data memory 71, a CT data memory 72, the processing circuitry 73, a display 74, a memory 75, and an input interface 76. The PET data memory 71, the CT data memory 72, the processing circuitry 73, the display 74, the memory 75, and the input interface 76 perform data communications with one another via, for example, a bus.

The PET data memory 71 is a storage device that stores single-event data and coincidence event data transferred from the PET gantry 10. The PET data memory 71 can be, for example, a hard disk drive (HDD), a solid state drive (SSD), or an integrated circuit storage device.

The CT data memory 72 is a storage device that stores CT raw data transferred from the CT gantry 30. The CT data memory 72 can be, for example, a HDD, an SSD, or an integrated circuit storage device.

The processing circuitry 73 includes, for example, hardware resources including a processor such as a CPU, an MPU, or a graphics processing unit (GPU), and memory such as a ROM and a RAM. The processing circuitry reads and executes various programs from the memory, to implement a reconstruction function 731, an image processing function 732, an imaging control function 733, an obtaining function 734, a generation function 735, a display control function 736, and a motion control function 737. In other words, the processing circuitry 73 corresponds to one or more processors that implement the functions corresponding to the respective programs by reading and executing the programs from the memory. Thus, after reading the programs, the processing circuitry 73 obtains the functions corresponding to the programs. The reconstruction function 731, the image processing function 732, the imaging control function 733, the obtaining function 734, the generation function 735, the display control function 736, and the motion control function 737 may be implemented by the processing circuitry 73 mounted on a single substrate or by the processing circuitry 73 mounted on two or more substrates in a distributed manner. The processing circuitry 73 implementing the reconstruction function 731, the image processing function 732, the imaging control function 733, the obtaining function 734, the generation function 735, the display control function 736, and the motion control function 737 correspond to a reconstruction unit, an image processing unit, an imaging control unit, an obtaining unit, a generator unit, a display control unit, and a motion control unit, respectively.

The processing circuitry 73 uses the reconstruction function 731 to reconstruct a PET image from coincidence event data. The PET image represents a distribution of positron-emitting radionuclides given to the subject P. The processing circuitry 73 also reconstructs a CT image from CT raw data. The CT image represents a spatial distribution of CT values as to the subject P. Any of existing image reconstruction algorithms including filtered back projection (FBP) and adaptive iterative reconstruction may be adoptable. The processing circuitry 73 can further generate PET localizer images from PET event data and CT localizer images from CT raw data.

The processing circuitry 73 uses the image processing function 732 to perform various kinds of image processing to the PET images and the CT images reconstructed by the reconstruction function 731. For example, the processing circuitry 73 subjects the PET images and the CT images to three-dimensional image processing including volume rendering, surface volume rendering, intensity projection, multi-planar reconstruction (MPR), and/or curved MPR (CPR) to generate display images.

For PET imaging, the processing circuitry 73 uses the imaging control function 733 to control the PET gantry 10 and the couch 50 in a synchronized manner. For CT imaging, the imaging control function 733 controls the CT gantry 30 and the couch 50 in a synchronized manner. For successive PET imaging and CT imaging, the imaging control function 733 controls the PET gantry 10, the CT gantry 30, and the couch 50 in a synchronized manner. The processing circuitry 73 can also cause the PET gantry 10 to perform a localizer scan (hereinafter, PET localizer scan) and the CT gantry 30 to perform a localizer scan (hereinafter, CT localizer scan). For a PET localization scan, the imaging control function 733 controls the PET gantry 10 and the couch 50 in a synchronized manner. For a CT localization scan, the imaging control function 733 controls the CT gantry 30 and the couch 50 in a synchronized manner.

The processing circuitry 73 uses the obtaining function 734 to obtain position information of each of the PET detector rings in the axial direction (Z-direction). For example, the obtaining function 734 obtains position information of each of the PET detector rings from an output from the rotary encoder attached to the pinion gears or an output from the rotary encoder attached to the motor rotational shaft in the drive mechanism. Alternatively, the position information may be obtained from an existing position sensor, in addition to the rotary encoder. The obtaining function 734 stores the position information in the memory 75. The obtaining function 734 further obtains information as to the subject P. The information as to the subject P refers to, for example, localizer images generated by a localizer scan of the subject P (CT localizer scan or PET localizer scan). The obtaining function 734 stores the information as to the subject P in the memory 75.

The processing circuitry 73 uses the generation function 735 to generate display information as to the positions of the PET detector rings 101 from the obtained position information. For example, the generation function 735 generates the display information containing the positions of the PET detector rings 101 and the information as to the subject P in association with each other. The display information represents, for example, a positional relationship between the PET detector rings 101 and the subject P lying on the table top 53. The display information includes ring objects individually indicating the positions of the PET detector rings 101. The display information may additionally include the position of the X-ray detector 32 relative to the subject P.

The display information may further include information indicating the scanning range of the subject P (such as a broken-line frame or a scan mode indicating a region of interest), in accordance with a user instruction input via the input interface 76 or a region to be examined included in an examination order output from a radiology information system (RIS) or a hospital information system (HIS).

The processing circuitry 73 uses the generation function 735 to select, in accordance with the scanning range input via the input interface 76, a scan mode close to the input scanning range (hereinafter, recommended mode) from multiple scan modes including a first mode and a second mode. The first mode is a scan mode in which the PET detector rings 101 are uniformly arranged at predetermined intervals according to the body length of the subject P. The first mode corresponds to, for example, the arrangement of the PET detector rings 101 as in the whole-body state WBS shown in FIG. 2. The second mode is a scan mode in which the PET detector rings 101 are densely arranged with respect to two or more individual regions of interest of the subject P in the axial direction. In the scanning range from the cervical region to the lumber region, the second mode corresponds to the arrangement of the PET detector rings 101 as in the partially dense state PDS shown in FIG. 2.

The processing circuitry 73 uses the display control function 736 to display the display information generated by the generation function 735 on the display 74. In response to a moving operation to any of the ring objects based on a user instruction input via the input interface 76, the display control function 736 updates the display information along with the motion of the ring object for display on the display 74. In response to a selection of the recommended mode from the scan modes by the generation function 735, the display control function 736 displays the selected scan mode (recommended mode) and a possible scanning range according to the recommended mode on the display 74, together with the display information. The possible scanning range is, for example, represented on the display information by the ring object arrangement corresponding to the recommended mode and/or a frame on the localizer image. The display forms of the display information will be described later in an information generation/display process.

The processing circuitry 73 uses the motion control function 737 to control the motion of the PET detector rings 101. For example, after completion of moving the ring object on the display 74, the motion control function 737 moves the PET detector ring corresponding to the moved ring object in the axial direction according to the moved ring object and the position information.

Specifically, in response to an instruction for completing moving the ring object input via the input interface 76, the motion control function 737 determines, based on the position of the moved ring object on the display information and the position information of the PET detector ring concerned, the position of the PET detector ring in the PET gantry 10, the position corresponding to the position of the moved ring object on the display information. The motion control function 737 then calculates a moving amount (for example, the rotation rate of the pinion gears or the motor) of the PET detector ring concerned according to the position information of the PET detector ring and the determined position. The motion control function 737 controls the ring moving mechanism 16 (specifically, the drive mechanism) to move the PET detector ring by the resultant amount.

The display 74 works to display various kinds of information including the display information generated by the generation function 735, under the control of the display control function 736 of the processing circuitry 73. Examples of the display 74 include a cathode ray tube (CRT) display, a liquid crystal display (LCD), an organic electroluminescence display (OELD), a plasma display, and any of other known displays in the related technical field. The display 74 may be a desktop type or may include a tablet terminal wirelessly communicable with the console device 70. The display 74 corresponds to a display unit.

The memory 75 is, for example, a storage device which stores various kinds of information, such as an HDD, an SSD, or an integrated circuit storage device. The memory 75 can be a driver that reads and writes various kinds of information from and to a portable storage medium as a compact disc (CD)-ROM drive, a digital versatile disc (DVD) drive, or a flash memory. The memory 75 stores, for example, various kinds of data related to the execution of the reconstruction function 731, the image processing function 732, the imaging control function 733, the obtaining function 734, the generation function 735, the display control function 736, and the motion control function 737. The memory 75 stores the position information obtained by the obtaining function 734. The memory 75 stores the display information generated by the generation function 735. The memory 75 stores the arrangement patterns of the PET detector rings 101 such as the first mode and the second mode. The memory 75 stores various kinds of computer programs for executing the reconstruction function 731, the image processing function 732, the imaging control function 733, the obtaining function 734, the generation function 735, the display control function 736, and the motion control function 737.

The input interface 76 serves to receive various kinds of user inputs (e.g., PET imaging instruction, CT imaging instruction, and scanning range selection), to convert the user inputs to electric signals for output to the processing circuitry 73. Examples of the input interface 76 include a mouse, a keyboard, a trackball, a switch, a button, a joystick, a touch-pad, and a touch panel display, as appropriate. In the present embodiment the input interface 76 is not limited to the one including any of such physical operational components as a keyboard, a trackball, a switch, a button, a joystick, a touch-pad, and a touch panel display. Other examples of the input interface 43 include electrical-signal processing circuitry that receives an electrical signal corresponding to an input from an external input device separated from the apparatus to output the electrical signal to the processing circuitry 73. Alternatively, the input interface 43 may include a tablet terminal wirelessly communicable with the console 40. The input interface 76 corresponds to an input unit.

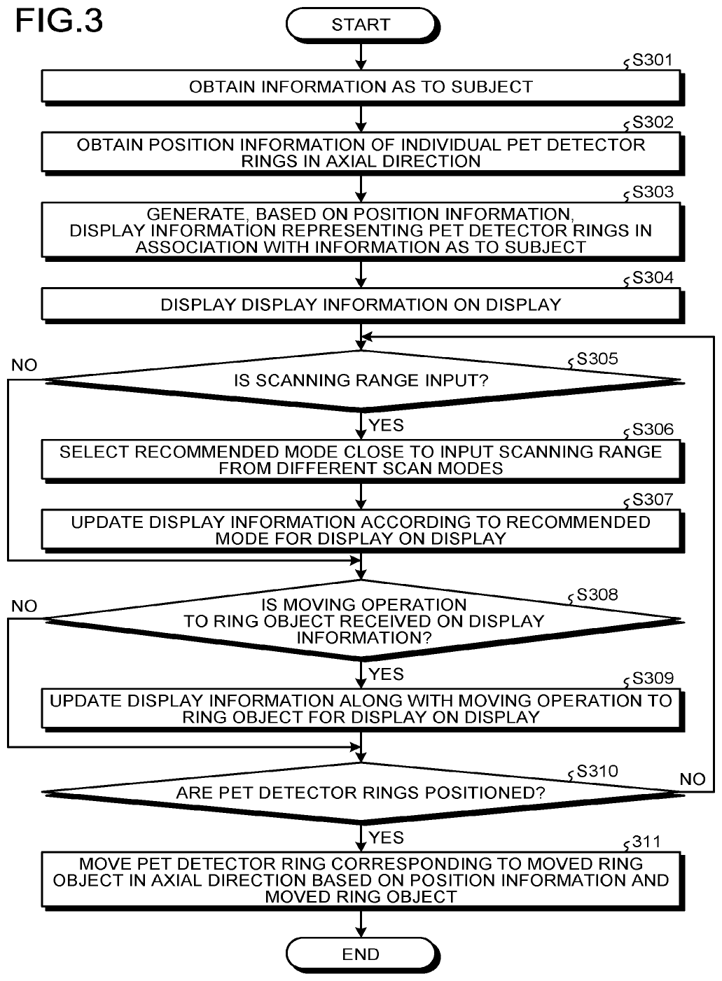

The overall features of the PET-CT apparatus 1 have been described. Hereinafter, an exemplary information generation/display process will be described with reference to FIG. 3 to FIG. 7. The information generation/display process is a process of generating display information as to the individual PET detector rings for display, based on position information of the individual PET detector rings 101. FIG. 3 is a flowchart illustrating an exemplary information generation/display process.

### Information Generation/Display Process

### Step S301

The processing circuitry 73 uses the obtaining function 734 to obtain information as to the subject P. For example, prior to S301, the processing circuitry 73 uses the imaging control function 733 to perform a localizer scan of the subject P (CT localizer scan or PET localizer scan). The processing circuitry 73 then uses the reconstruction function 731 or the image processing function 732 to generate a localizer image of the subject P. The processing circuitry 73 then uses the obtaining function 734 to obtain the localizer image as information as to the subject P at S301.

### Step S302

The processing circuitry 73 uses the obtaining function 734 to obtain position information of the individual PET detector rings in the axial direction. The obtaining function 734 obtains the position information of each of the PET detector rings from the output of each rotary encoder in the ring moving mechanism 16, for example.

### Step S303

The processing circuitry 73 uses the generation function 735 to generate, based on the position information, display information as to the PET detector rings 101 representing the PET detector rings 101 in association with the information as to the subject P. Specifically, the generation function 735 superimposes the ring objects on the localizer image being information as to the subject P at positions based on the position information, to generate display information. The generation function 735 may further add, to the display information, a frame representing the table top 53 on the back side of the localizer image of the subject P. Alternatively, the generation function 735 may generate display information by additionally superimposing a frame representing the PET gantry 10 and/or the CT gantry 30, and a frame representing the X-ray detector 32 on the localizer image based on the position information. The generation function 735 may generate display information, using the profile of the localizer image as the information as to the subject P, in place of the localizer image of the subject P.

### Step S304

The processing circuitry 73 uses the display control function 736 to display the display information on the display 74. The ring objects are individually movable on the display information in the axial direction (i.e., the long axis direction of the table top 53) in accordance with a user instruction input via the input interface 76, when appropriate.

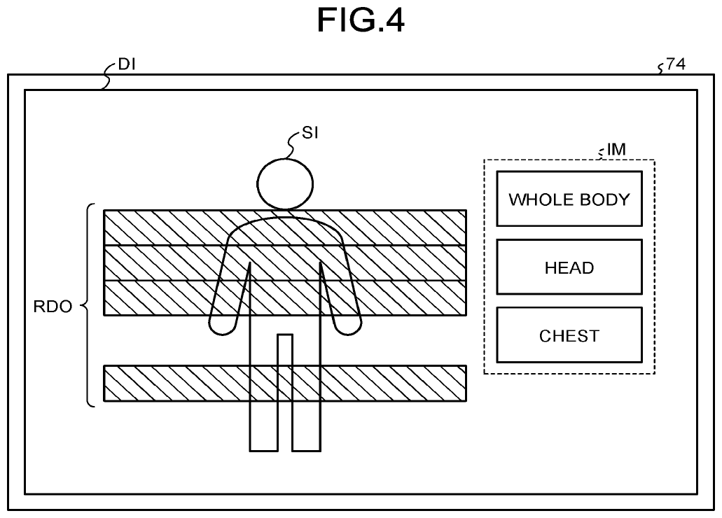

FIG. 4 depicts exemplary display information DI displayed on the display 74. As illustrated in FIG. 4, the display information DI includes a localizer image SI of the subject P, four ring objects RDO corresponding to four PET detector rings, and a scan mode IM. As illustrated in FIG. 4, the four ring objects RDO are superimposed on the localizer image SI of the subject P in accordance with the position information. As illustrated in FIG. 4, displaying the four ring objects RDO corresponds to having the positions of the PET detector rings 101 reflected on the display information DI.

### Step S305

In response to an input of a scanning range via the input interface 76 (Yes at step S305), the processing circuitry 73 performs operation at step S306. Without a scanning range input (No at step S305), the processing circuitry 73 performs operation at step S308.

### Step S306

The processing circuitry 73 uses the generation function 735 to select a scan mode (recommended mode) close to the input scanning range from different scan modes. As an example, the user may swipe the localizer image SI of the subject P on the display information DI craniocaudally. In such a case, the generation function 735 determines the scanning range from the swipe range and the craniocaudal length of the subject P on the localizer image SI. In addition to the swipe, the scanning range can be input through any of known operations, for example, using a mouse to craniocaudally move a cursor by a click or to rotate the scroll wheel while clicking the mouse. The generation function 735 determines the scan mode corresponding to the input scanning range as a recommended mode. The recommended mode may be a scan mode selected by the user from multiple scan modes IM on the display information DI. Note that the memory 75 pre-stores the positions of the ring objects and the positions of the PET detector rings 101 in the respective scan modes in association with each other.

### Step S307

The processing circuitry 73 uses the display control function 736 to update the display information DI in accordance with the recommended mode for display on the display 74. As an example, the display control function 736 displays the selected scan mode (recommended mode) and a possible scanning range according to the recommended mode on the display 74 together with the display information. Specifically, the display control function 736 displays the recommended scan mode IM among the different scan modes IM in a highlighted manner on the display information DI. The display control function 736 also displays the ring objects RDO at positions according to the recommended mode on the display information DI. The positions of the ring objects RDO according to the recommended mode correspond to, for example, a possible PET scanning range. The possible scanning range is not limited to the positions of the ring objects RDO according to the recommended mode and it can be displayed as a frame on the localizer image SI, for example.

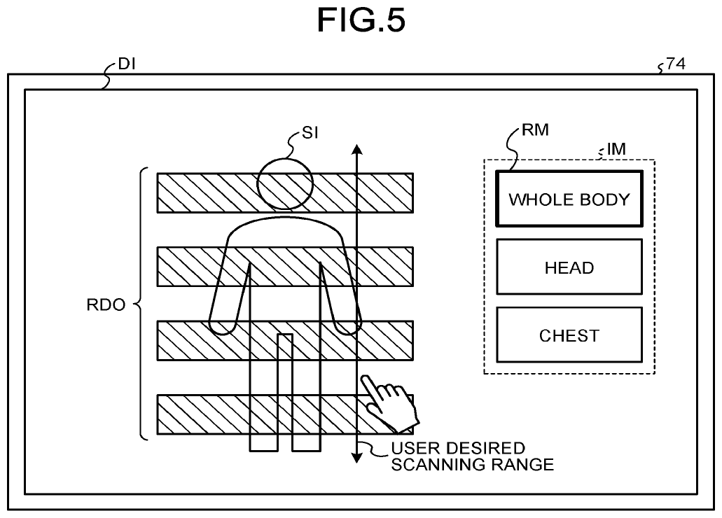

FIG. 5 depicts exemplary display information DI representing that the localizer image SI of the subject P is being swiped from the head region to the leg region at step S306. The swiped head to leg region of the subject P corresponds to a user desired scanning range as illustrated in FIG. 5. Among the different scan modes IM, the scan mode “whole body” being a recommended mode (first mode) RM is displayed in a highlighted manner on the display information DI, as illustrated in FIG. 5. In addition the ring objects RDO are displayed at the positions according to the first mode on the display information DI, as illustrated in FIG. 5.

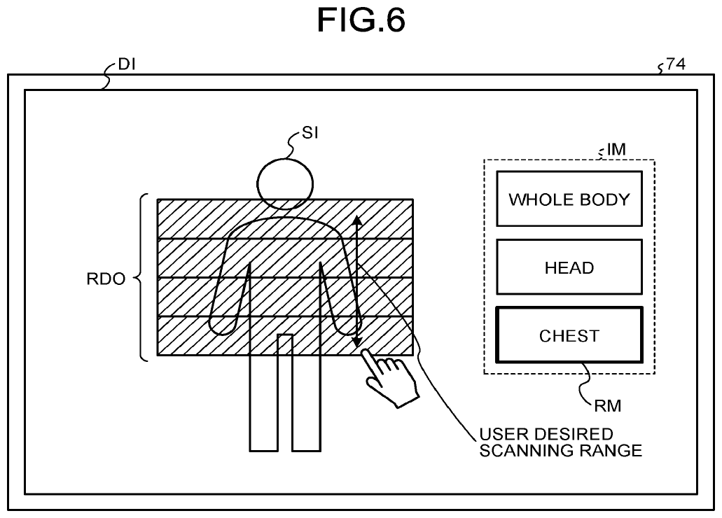

FIG. 6 depicts exemplary display information DI representing that the localizer image SI of the subject P is being swiped from the shoulder region to the lumber region at step S306. The swiped shoulder to lumber region of the subject P corresponds to a user desired scanning range as illustrated in FIG. 6. Among the different scan modes IM, the scan mode “chest” being a recommended mode (second mode) RM is displayed in a highlighted manner on the display information DI, as illustrated in FIG. 6. In addition the ring objects RDO are displayed at the positions according to the second mode on the display information DI, as illustrated in FIG. 6.

### Step S308

In response to a receipt of a moving operation to the ring objects via the input interface 76 (Yes at step S308), the processing circuitry 73 performs operation at step S309. Without a receipt of a moving operation to the ring objects (No at step S308), the processing circuitry 73 performs operation at step S310.

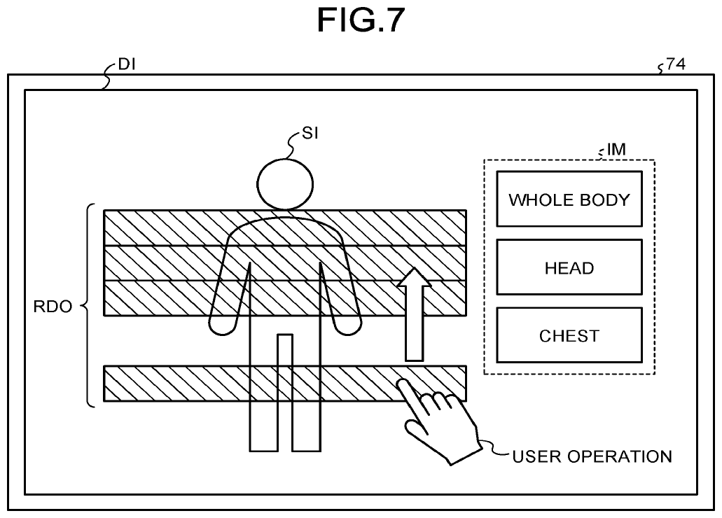

FIG. 7 depicts an exemplary user operation with respect to the display information DI of FIG. 4. As illustrated in FIG. 7, the ring object located at the knee position on the localizer image SI is movable by a user operation. As an example, FIG. 7 shows a user operation to move the ring object located at the knee position on the localizer image SI in the direction indicated by the arrow. That is, the processing circuitry 73 uses the display control function 736 to update the display information DI along with the moving operation to the ring object for display on the display 74.

### Step S309

The processing circuitry 73 uses the display control function 736 to update the display information along with the moving operation to the ring object for display on the display 74. For example, in FIG. 7 the ring object located at the knee position is moved to the ring object located at the abdominal position on the localizer image SI by a user operation. In this case the positional relationship between the ring objects and the localizer image SI is changed to the one as shown in FIG. 6.

### Step S310

After the positions of the PET detector rings 101 are set by the user instruction input via the input interface 76 (Yes at step S310), the processing circuitry 73 performs operation at step S311. Without the positions of the PET detector rings 101 set (No at step S310), the processing circuitry 73 repeats the operations at step S305 and subsequent steps. The operations at step S307 and S309 correspond to presenting demonstration video representing the movement of the PET detector rings 101 to the user.

### Step S311

The processing circuitry 73 uses the motion control function 737 to move the PET detector ring corresponding to the moved ring object in the axial direction based on the position information and the moved ring object. Specifically, the motion control function 737 feeds back the positions of the ring objects to the positions of the PET detector rings 101. The processing circuitry 73 may use the display control function 736 to update the display information DI for display on the display 74 in accordance with the change in the position information due to the motion of the PET detector rings. Thus, at S311 the display control function 736 may display the display information DI representing the ring objects moved in conjunction with the motion of the PET detector rings, on the display 74. After completion of the information generation/display process, the subject P is subjected to PET imaging by a user instruction given via the input interface 76.

The PET-CT apparatus (or PET apparatus) 1 according to the first embodiment described above obtains position information of each of the PET detector rings in the axial direction of the bore 20 into which the table top 53 having the subject P lying thereon is inserted. The PET detector rings are movable with respect to the table top 53 in the axial direction. The PET-CT apparatus 1 generates display information DI as to the positions of the PET detector rings 101 based on the obtained position information and displays the display information DI on the display 74. In the present embodiment the PET-CT apparatus 1 may additionally obtain information as to the subject P to generate display information DI containing the positions of the PET detector rings 101 and the information as to the subject P in association with each other. Further, in the present embodiment the PET-CT apparatus 1 allows a moving operation to the ring objects representing the individual PET detector rings on the display information DI.

In addition, after the positions of the ring objects are set, the PET-CT apparatus (or PET apparatus) 1 according to the first embodiment moves the PET detector ring corresponding to the moved ring object in the axial direction based on the moved ring object and the position information, and updates the display information DI along with the moving operation to the ring object for display on the display 74.

As such, the PET-CT apparatus (or PET apparatus) 1 according to the first embodiment can reflect the positions of the PET detector rings 101 on the information on the display 74, thereby allowing the user to operate the PET detector rings 101 on the display 74 to change their positions. Further, in the present embodiment the PET-CT apparatus 1 can display the ring objects representing the positions of the PET detector rings 101 on the localizer image resulting from a CT or PET localizer scan in a superimposed manner on the user operable display 74.

In this manner, the PET-CT apparatus (or PET apparatus) 1 according to the first embodiment allows the user to change the ring objects in position on the display 74 in line with his or her desired scanning range to be able to display a possible scanning range together with a demonstration of the PET detector rings 101 in motion. Thus, after the user sets the positions or coordinates of the ring objects, the PET-CT apparatus 1 can feed back the set positions to the control over the PET detector rings 101 to move the PET detector rings 101 to the user set positions.

Owing to such features, the PET-CT apparatus (or PET apparatus) 1 according to the first embodiment enables the user to easily know the positions of the PET detector rings 101 that are movable relative to the table top 53, and to easily adjust the arrangement of the PET detector rings 101. Thus, the PET-CT apparatus 1 of the present embodiment can contribute to lessening users' burdens on the operation of the PET detector rings 101 and improving throughput of examination of the subject P. In addition, the PET-CT apparatus 1 of the present embodiment can maximize clinical efficacy of the PET scanning using the two or more movable PET detector rings 101 and enhance the image quality of the images resulting from the PET scanning.

Further, the PET-CT apparatus (or PET apparatus) 1 according to the first embodiment receives an input of a scanning range of the subject P on the display information DI to select a scan mode close to the input scanning range from the different scan modes including the first mode and the second mode and display, on the display 74, the selected scan mode (recommended mode) and a possible scanning range according to the recommended mode together with the display information DI.

Thus, the PET-CT apparatus (or PET apparatus) 1 according to the first embodiment prepares multiple scan modes including the first mode and the second mode in software or in the memory 75 to be able to determine the scan mode closest to a user selected scanning range from the multiple scan modes. Thereby, the PET-CT apparatus 1 can propose a scan mode to the user while presenting a possible scanning range. As such, the PET-CT apparatus 1 of the present embodiment can shorten the length of time for the user to select the scanning range and adjust the positions of the PET detector rings 101, which results in improving an examination work flow with respect to the subject P in efficiency.

### First Application

In the information generation/display process of a first application, the processing circuitry 73 generates a count-rate map for each of the PET detector rings 101 from coincidence event data and displays the count-rate map on the ring objects RDO in a superimposed manner on the display information DI displayed on the display 74. The count-rate map represents count rates of gamma rays per unit time in the multiple PET detectors. In the first application, the count rate is generated during PET imaging of the subject P, therefore, it may be referred to as real-time count rate. Specifically, the count rate is generated during a PET scan and/or a PET localizer scan and displayed on the display information DI. The unit time may be preset as 30 seconds or one minute or may be optionally set by a user instruction given via the input interface 76.

The processing circuitry 73 uses the obtaining function 734 to obtain coincidence event data based on the output of the multiple gamma ray detectors (PET detectors) 17 included in the individual PET detector rings. For example, the obtaining function 734 obtains coincidence event data from the coincidence circuitry 15 during a PET scan of the subject P.

The processing circuitry 73 uses the generation function 735 to generate a count-rate map for each of the PET detector rings based on the coincidence event data during a PET scan of the subject P. The count-rate map is, for example, represented as a planar image generated by adding (compressing) coincidence-event count rates of two vertically opposing PET detectors in the PET detector rings. Each of the pixels in the count-rate map represents a position of a PET detector and each pixel value represents a brightness value (or a color value) indicating a count rate. The count-rate map is not limited to a planar image and may be, for example, represented as a three-dimensional image representing count rates in units of PET detectors, i.e., in units of detection channels according to volume data of the subject P.

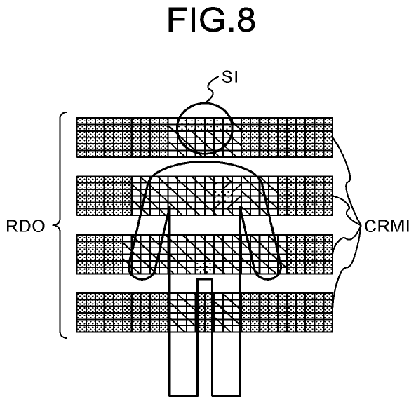

The processing circuitry 73 uses the display control function 736 to display, on the display 74, display information DI showing count-rate maps on the ring objects representing the PET detector rings in a superimposed manner. FIG. 8 depicts an example of count-rate maps CRMI displayed on the display 74. As illustrated in FIG. 8, four count-rate maps CRMI corresponding to four PET detector rings are individually superimposed on four ring objects RDO representing the four PET detector rings. In FIG. 8 the grids of the count-rate maps CRMI correspond to the PET detectors, i.e., channels one to one. In FIG. 8, differences in count rate on the count-rate maps CRMI are indicated by hatching, however, in reality the magnitude of the count rates is represented by differences in brightness value.

The PET-CT apparatus (or PET apparatus) 1 according to the first application of the first embodiment as described above obtains coincidence event data based on the output from the PET detectors individually included in the PET detector rings to generate, for each of the PET detector rings, a count-rate map CRMI representing count rates of gamma rays in the PET detectors per unit time from the coincidence event data. The PET-CT apparatus 1 then displays, on the display 74, display information DI showing that the count-rate maps CRMI are superimposed on the ring objects individually representing the PET detector rings.

Owing to such features, the PET-CT apparatus (or PET apparatus) 1 according to the first application of the first embodiment can measure the count rates in real time from coincidence events which are counted in each channel of the gamma-ray detectors 17 during data acquisition in the PET imaging, and display the count rates together with the display information DI on the display 74 in real time. According to the first application, thus, the user can see or check accumulation and a dynamic distribution of radionuclides inside the subject P in real time.

As such, the PET-CT apparatus (or PET apparatus) 1 according to the first application of the first embodiment enables the user to select the PET scanning range based on, for example, the count rates. Thereby, the user can select the scanning range correctly and proceed to a main scan (PET scan) with a higher dose in the PET imaging. According to the first application, thus, the user can perform a PET scan of the subject P without a CT localizer scan, which can reduce exposure of the subject P to radiation. In addition, the PET-CT apparatus 1 of the first application can display the count rates in real time, which results in shortening a PET examination time using fluorodeoxyglucose (FDG) as radionuclides, that is, shortening or improving an examination work flow, for example. Thus, the PET-CT apparatus 1 of the first application can decrease an applied dose of radionuclides to the subject P, leading to decreasing exposure of the subject P to radiation.

### Second Application

In the information generation/display process of a second application, the processing circuitry 73 performs PET imaging (first scan) of the subject P while the PET detector rings 101 are uniformly arranged with predetermined intervals based on the body length of the subject P (first mode) to thereby generate an accumulation map for each of the PET detector rings. The accumulation map represents an accumulation distribution of gamma-ray counts. According to the second application, the processing circuitry 73 then defines, as a recommended arrangement of the PET detector rings 101 for the second scan, a dense arrangement of the PET detector rings 101 about the position of a PET detector ring with a least accumulation among the accumulation distributions of the PET detector rings 101 and displays the recommended arrangement on the display information DI displayed on the display 74.

The processing circuitry 73 uses the imaging control function 733 to perform a first scan (PET scan) of the subject P in the first mode. In the first scan, the processing circuitry 73 uses the obtaining function 734 to obtain coincidence event data from the output of the PET detectors individually included in the PET detector rings.

The processing circuitry 73 uses the generation function 735 to generate, for each of the PET detector rings, an accumulation map representing an accumulation distribution of gamma-ray counts, based on the coincidence event data. The accumulation map is, for example, dynamically generated during the first scan. The accumulation map corresponds to a map indicating a sum of the count values of the respective PET detectors from a start of the first scan to a current point in time for each of the PET detector rings.

The processing circuitry 73 uses the display control function 736 to display, on the display 74, display information DI representing that accumulation maps are superimposed on the corresponding ring objects. After completion of the first scan, the display control function 736 identifies an accumulation map with a least accumulation from among the multiple accumulation maps on the display information DI. The display control function 736 sets, as a recommended arrangement, an arrangement of the PET detector rings 101 that the PET detector rings 101 are densely arranged about the position of the PET detector ring corresponding to the identified accumulation map (hereinafter, referred to as densely arranged state) and displays the recommended arrangement on the display 74. The recommended arrangement is an arrangement for the second scan executable after the first scan.

The processing circuitry 73 recognizes a user's acceptance of the densely arranged state in the second scan from a user instruction given via the input interface 76, and then uses the motion control function 737 to perform control over the ring moving mechanism 16 to place the PET detector rings 101 in the densely arranged state.

In the first scan of the first mode, the PET-CT apparatus (or PET apparatus) 1 according to the second application of the first embodiment as described above obtains coincidence event data based on the output from the PET detectors individually included in the PET detector rings, to generate an accumulation map for each of the PET detector rings from the coincidence event data and display, on the display 74, the display information representing the densely arranged state of the PET detector rings 101 as a recommended arrangement for the second scan.

Owing to such features, the PET-CT apparatus (or PET apparatus) 1 according to the second application of the first embodiment can efficiently acquire coincidence event data to obtain uniform PET images of the whole body of the subject P. Thus, the PET-CT apparatus 1 of the second application can improve the work flow of PET examination of the subject P, improving examination efficiency (throughput).

### Third Application

In the information generation/display process of a third application, the processing circuitry 73 displays a switch object on the display 74 located, for example, on the surface of the PET gantry 10 and/or the CT gantry 30. The switch object represents a switch from the first scan (for example, PET localizer scan) to the second scan (for example, main PET scan), triggered by the event that the count of the count rates on the count-rate map RDO has reached a predetermined value. The count-rate map RDO is generated in the same manner as in the first application, therefore, a detailed description thereof is omitted herein. The count-rate map RDO is displayed on the display 74 during the first scan and during the second scan.

The memory 75 stores therein the predetermined value. The predetermined value may be appropriately settable or variable in accordance with a user instruction given via the input interface 76. Alternatively, the predetermined value may be set depending on a type of radionuclides.

The processing circuitry 73 uses the display control function 736 to determine whether or not the count of the count rates on the count-rate map RDO has reached the predetermined value. Another function of the processing circuitry 73 may perform the determination or an additional determining function to perform the determination may be included in the processing circuitry 73. The processing circuitry 73 implementing the determining function corresponds to a determiner unit. The display control function 736 displays the switch object on the display 74, triggered by the event that the count has reached the predetermined value. The switch object represents information for presenting or proposing the user to switch from a PET localizer scan to a main PET scan. Examples of the switch object include text information such as “Perform a Main Scan?” and/or a button display such as a “main scan start” button.

In response to a receipt of a user instruction to switch the scan mode via the input interface 76, the processing circuitry 73 uses the motion control function 737 to perform control over the ring moving mechanism 16 to move the PET detector rings 101 in the axial direction so as to make a transition from the first mode for the first scan to the second mode according to a region of interest of the subject P to be scanned. Thereby, the arrangement of the PET detector rings 101 changes from the first-mode arrangement to the second-mode arrangement. The processing circuitry 73 then uses the imaging control function 733 to perform a second scan of the subject P.

The PET-CT apparatus (or PET apparatus) 1 according to the third application of the first embodiment as described above displays display information DI containing the count-rate map RDO on the display 74 located on the surface of the PET gantry 10 and/or the CT gantry 30. Such a display allows the user to check the count rates in the first scan, as in the first application. The PET-CT apparatus of the third application also displays the switch object on the display 74, triggered by the event that the count on the count-rate map RDO has reached the predetermined value. In addition, the PET-CT apparatus of the third application performs a main PET scan of the subject P in response to the operation to the switch object.

Owing to such features, the PET-CT apparatus (or PET apparatus) 1 according to the third application of the first embodiment allows the user to see the count-rate map RDO in real time and switch the scan mode from the first scan to the main PET scan (second scan) for acquisition of coincidence event data through the operation from the PET gantry 10 and/or the CT gantry 30. Thus, when applying short-half-life radionuclides such as rubidium 82 (82Rb) to the subject P, the PET-CT apparatus 1 of the third application can allow the user to easily check a dynamic distribution (count-rate map RDO) of the short-half-life radionuclides in real time and easily ensure administration of the short-half-life radionuclides to the subject P, for example. Further, the PET-CT apparatus 1 of the third application allows the user to start scanning the subject P after confirming in real time that the subject P has been given the short-half-life radionuclides. As such, the PET-CT apparatus 1 of the third application can reduce failures in performing the main PET scan of the subject P and decrease the scan switch timing in comparison with the conventional apparatuses, thereby improving the work flow of PET examination of the subject P and improving examination efficiency (throughput).

### Fourth Application

In a fourth application, in a PET scan of the subject P the processing circuitry 73 performs the first scan while the PET detector rings 101 are arranged in the first mode, and after the first scan, moves, for the second scan, the PET detector rings 101 or the table top 53 on which the subject P is lying in the axial direction so that the PET detector rings 101 are arranged at positions corresponding to predetermined intervals in the first mode.

In the first scan of the subject P, the processing circuitry 73 uses the motion control function 737 to move the PET detector rings 101 in the axial direction to uniformly place the PET detector rings 101 with predetermined intervals according to the body length of the subject P. In other words, the motion control function 737 controls the ring moving mechanism 16 to implement the first-mode arrangement of the PET detector rings 101. Thereby, the PET detector rings 101 can be arranged according to the first mode prior to an execution of the first scan.

After the first scan, the processing circuitry 73 uses the motion control function 737 to move the PET detector rings 101 or the table top 53 on which the subject P is lying in the axial direction so that the PET detector rings 101 are arranged at positions corresponding to the predetermined intervals in the first mode. Specifically, the motion control function 737 controls the ring moving mechanism 16 or the couch driver 54 to place the PET detector rings 101 individually at positions corresponding to the predetermined intervals at which every two adjacent PET detector rings 101 are arranged in the first mode.

The processing circuitry 73 uses the display control function 736 to display, on the display 74, display information DI representing the arrangements of the PET detector rings 101 in the first scan and in the second scan after the first scan. Specifically, the display control function 736 displays on the display 74 display information DI representing different relative positions between the PET detector rings 101 and the table top 53 during the first scan and during the second scan. Namely, the display control function 736 displays the display information DI on the display 74 such that the ring objects representing the positions of the PET detector rings 101 during the first scan and the ring objects during the second scan are alternating with each other on the localizer images.

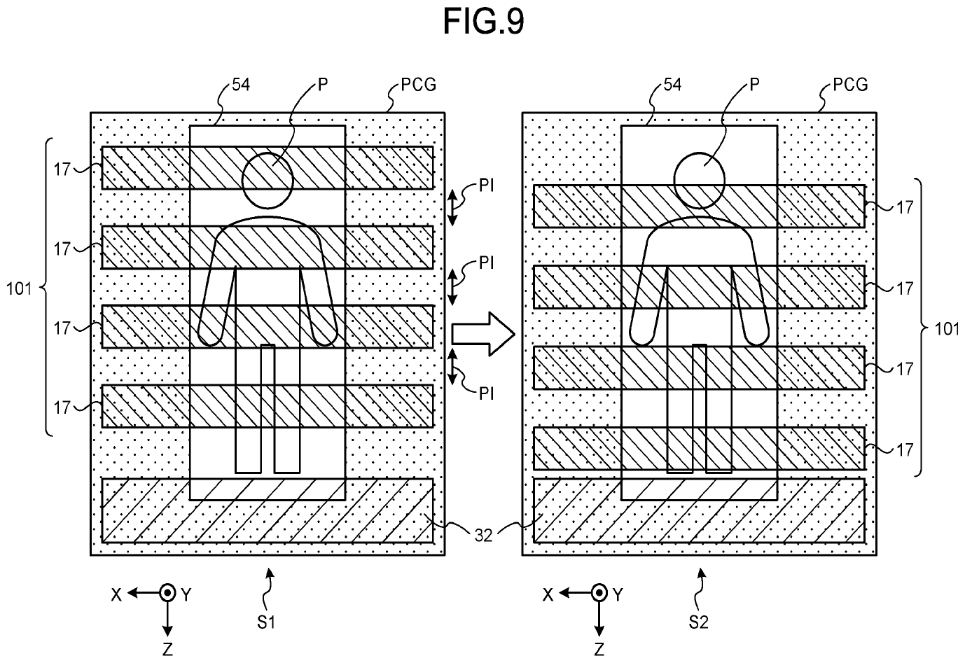

FIG. 9 illustrates differences between a first scan S1 and a second scan S2 in terms of the arrangement of the PET detector rings 101 relative to the subject P, by way of example.

In FIG. 9 the PET gantry 10 and the CT gantry 30 are represented by the same housing (PET/CT gantry) PCG. As illustrated in FIG. 9, in the first scan S1 the PET detector rings 101 are uniformly arranged with predetermined intervals PI so as to image the whole body of the subject P. In the second scan S2, as illustrated in FIG. 9, the PET detector rings 101 are arranged at respective positions corresponding to the predetermined intervals PI so as to image the whole body of the subject P. The display control function 736 may display, on the display 74, the display information DI showing the ring objects RDO representing the respective PET detector rings 101 on the localizer image of the subject P, as illustrated in FIG. 9, prior to the first scan and prior to the second scan.

In the first scan, the PET-CT apparatus (or PET apparatus) 1 according to the fourth application of the first embodiment as described above moves the PET detector rings 101 in the axial direction to implement the first mode. After the first scan, the PET-CT apparatus 1 moves the PET detector rings 101 or the table top 53 in the axial direction to place the PET detector rings 101 at the respective positions corresponding to the predetermined intervals PI of the first mode. The PET-CT apparatus 1 displays the display information DI showing the PET detector rings 101 and the table top 53 in different positional relations on the display 74 during the first scan and during the second scan, as illustrated in FIG. 9.

Owing to such features, after performing a first PET scan of the whole body of the subject P with the PET detector rings 101 uniformly arranged, the PET-CT apparatus (or PET apparatus) 1 according to the fourth application of the first embodiment can perform positional adjustment of the PET detector rings 101 or the table top 53 in the period between the first PET scan and a second PET scan such that in the second PET scan, the PET detector rings 101 are individually placed to fill the spacing PI in-between every two adjacent PET detector rings set in the first PET scan. According to the fourth application, thus, the PET-CT apparatus 1 can scan a wider scanning range of the subject P such as the whole body at higher sensitivity. Consequently, the PET-CT apparatus 1 of the fourth application can efficiently acquire PET event data to obtain higher-sensitivity, whole-body images, leading to improving the work flow of PET examination of the subject P.

### Modification

The features of this medication are in using no localizer image as information as to the subject P. Specifically, in this modification the obtaining function 734 obtains position information and information as to the subject P from video data generated by a camera. The camera is, for example, provided outside the PET gantry 10 and/or in the CT gantry 30. The obtaining function 734 also obtains the information as to the subject P from the video data.

The PET detector rings each include a projector capable of projecting light orthogonally relative to the table top. Each of the PET detector rings incorporates the projector in opposition to the table top 53, for example. The projector projects laser beams to the table top orthogonally, i.e., in parallel to the vertical direction. The projector is not limited to a laser projecting device. In the case of using an optical camera, for example, the projector may generate visible light. In the case of using an infrared camera, the projector may generate infrared light. The projector may be controlled to turn ON/OFF for light projection in accordance with a user instruction given via the input interface 76, for example.

The processing circuitry 73 uses the obtaining function 734 to obtain, as position information, projection positions of the laser beams vertically projected to the table top 53 on which the subject P is lying from the individual projectors included in the PET detector rings 101. For example, the obtaining function 734 identifies the projection positions on the video data output from the camera through image processing and obtains the identified projection positions as position information. The obtaining function 734 also identifies a subject-P area on the table top 53 through image processing to the video data to obtain the identified subject-P area as information as to the subject P. The image processing to the video data output from the camera may be performed by the image processing function 732. The image processing for identifying the projection positions is feasible by any of known techniques, therefore, an explanation thereof is omitted herein.

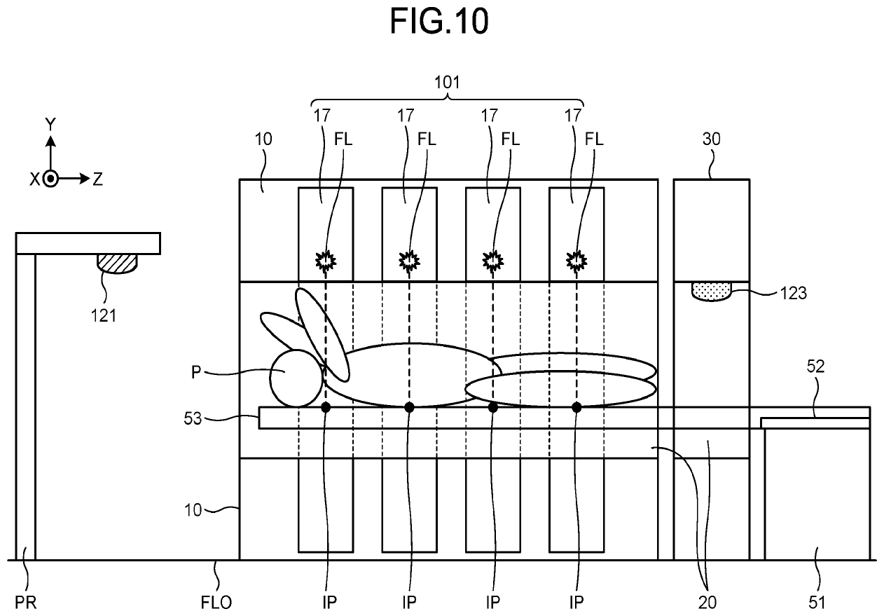

FIG. 10 illustrates projection positions IP by the laser beams projected from projectors FL, by way of example. As illustrated in FIG. 10, the PET detector rings 101 are uniformly arranged with predetermined intervals in accordance with the body length of the subject P (first mode). As illustrated in FIG. 10, the laser is projected in parallel to the vertical direction toward the table top 53 from the projectors FL and reaches the table top 53 and the projection positions IP corresponding to the PET detector rings 101. Outside the PET gantry 10, a first camera 121 is, for example, installed on the beam of a support post PR standing on the floor FLO of the examination room, as illustrated in FIG. 10. A second camera 123 is also installed, for example, above the bore 20 of the CT gantry 30. In this modification the installation locations of the cameras are not limited to the ones illustrated in FIG. 10, and can be set to any locations as long as the cameras can capture the projection positions IP. In this modification the video data is output from the cameras to the console 70 in a wired or wireless manner. The video data is stored in the memory 75.

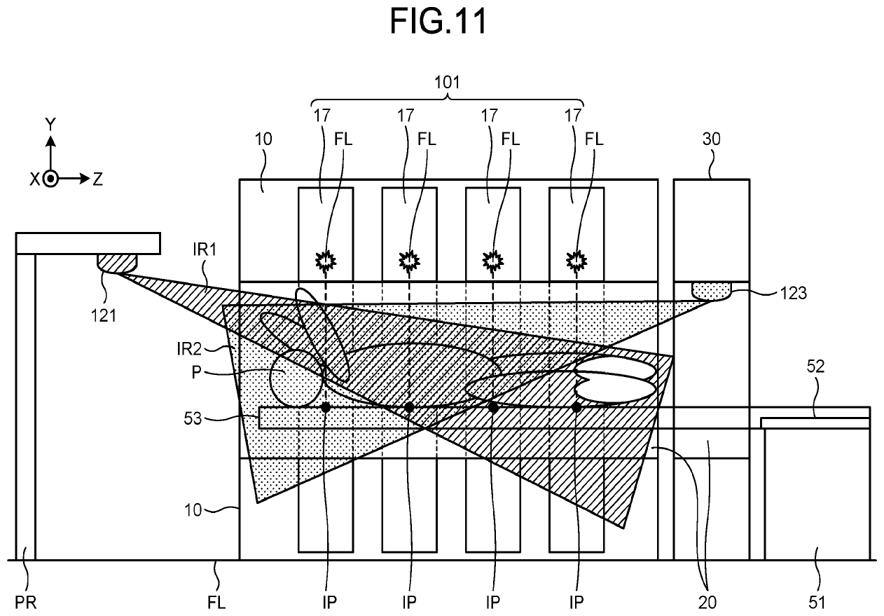

FIG. 11 illustrates an imaging area IR1 of the first camera 121 and an imaging area IR2 of the second camera 123 by way of example. As illustrated in FIG. 11, the first camera 121 and the second camera 123 can capture all the projection positions IP. The processing circuitry 73 uses the obtaining function 734 to obtain, as position information, positions of the PET detector rings 101 relative to the table top 53 from the projection positions IP captured on the video data output from the first camera 121 and the second camera 123.

The PET-CT apparatus (or PET apparatus) 1 according to this modification of the first embodiment as described above obtains, as position information, the projection positions IP of the laser beams projected vertically to the table top 53 from the projectors FL individually included in the PET detector rings 101. According to this modification, thus, the first camera 121 located outside the PET gantry 10 and the second camera 123 installed in the CT gantry 30 can capture the subject P contained in the PET gantry 10 and the laser projected positions IP. In addition, the PET-CT apparatus 1 of this modification performs image processing to the captured video, thereby allowing the user to see or check the position of the subject P and the positions of the PET detector rings 101 on the live-action image in real time.

As such, the PET-CT apparatus (or PET apparatus) 1 according to this modification of the first embodiment can obtain the position information of the PET detector rings 101 and the information as to the subject without performing a localizer scan for obtaining localizer images. Further, as explained with reference to FIG. 5 and FIG. 6 in the first embodiment, the PET-CT apparatus 1 of this modification includes the function of proposing to the user a preset scan mode that allows the PET detector rings 101 to be arranged in accordance with a user selected scanning range (e.g., FIG. 5 and FIG. 6). By combining such a function and this modification, the PET-CT apparatus 1 can propose, to the user, the optimal arrangement of the PET detector rings 101 with respect to a user desired scanning range without performing a localizer scan of the subject P.

Owing to such features, the PET-CT apparatus 1 of this modification can reduce the exposure of the subject P to radiation for obtaining localizer images. Further, the PET-CT apparatus 1 of this modification can abate users' trouble in selecting the scanning range, resulting in improving the work flow of examination of the subject P in efficiency and improving examination efficiency (throughput).

### Second Embodiment

The features of a second embodiment are in moving the PET detectors to outside the X-ray irradiation range prior to CT imaging. For example, the processing circuitry 73 uses the motion control function 737 to control the ring moving mechanism 16 to move the PET detectors to outside the X-ray irradiation range prior to CT imaging.

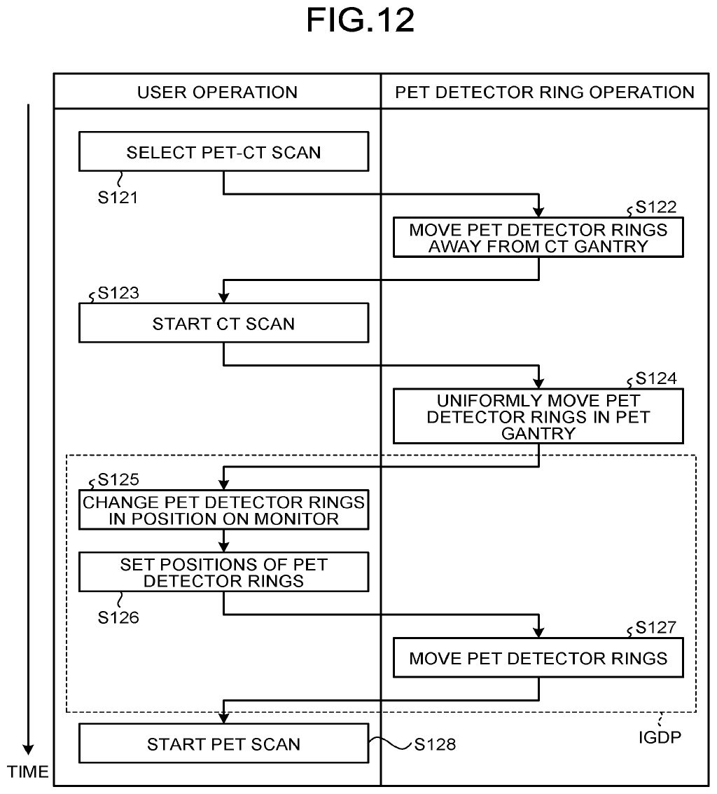

FIG. 12 is a flow diagram illustrating an exemplary operation of the PET detector rings 101 in a PET-CT examination. FIG. 12 illustrates an example that CT scanning precedes PET scanning. Alternatively, CT scanning may succeed PET scanning. As illustrated in FIG. 12, the input interface 76 receives a user instruction for selecting a PET-CT scan, i.e., an instruction for performing a CT scan (S121).

Triggered by the receipt of the CT scanning instruction, the processing circuitry 73 uses the motion control function 737 to perform control over the ring moving mechanism 16 to evacuate the PET detector rings 101 from the irradiation range of the CT scan. Thereby, the PET detector rings 101 are moved away from the CT gantry 30 (S122). The processing circuitry 73 may use the display control function 736 to display display information DI showing that the PET detector rings 101 are being evacuated to outside the X-ray irradiation range, on the display 74.

After the PET detector rings 101 are evacuated to outside the X-ray irradiation range, the processing circuitry 73 uses the imaging control function 733 to perform a CT scan of the subject P (S123). Alternatively, the imaging control function 733 may perform a CT localizer scan of the subject P before a CT scan.

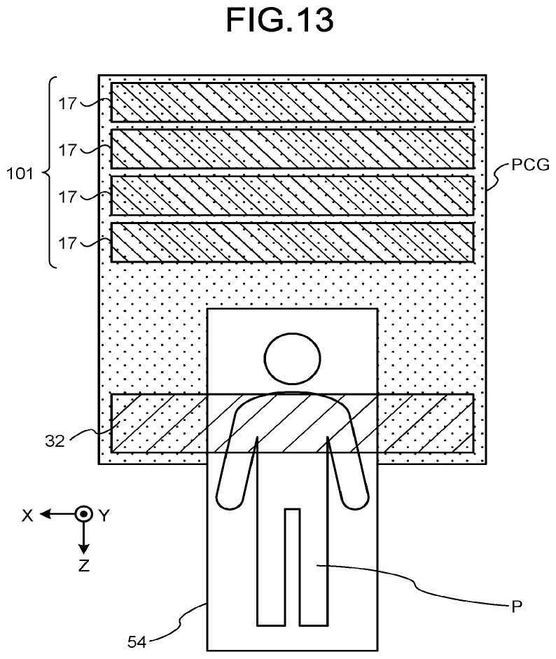

FIG. 13 illustrates the position of the PET detector rings 101 evacuated outside the X-ray irradiation range by way of example. As illustrated in FIG. 13, the PET detector rings 101 are located outside the X-ray irradiation range during X-ray irradiation to the subject P. The display control function 736 may display the diagram of FIG. 13 as display information DI on the display 74 at S122, for example.

After the CT scan of the subject P, the processing circuitry 73 uses the motion control function 737 to control the ring moving mechanism 16 so as to uniformly arrange the PET detector rings 101 in line with the body length of the subject P in the long axis direction of the table top 53 inside the PET gantry 10 (S124). This completes the arrangement of the PET detector rings 101 in the first mode.

After the PET detector rings 101 are arranged in the first mode, the PET detector rings 101 are changed in position on the monitor (display 74) by a user operation (S125). The positions of the PET detector rings are decided (S126).

The PET detector rings are then moved (S127). The processing from S125 to S127 is similar to or the same as the information generation/display process IGDP of the first embodiment, therefore, a description thereof is omitted herein. After completion of moving the PET detector rings, the processing circuitry 73 uses the imaging control function 733 to perform a PET scan of the subject (S128).

The PET-CT apparatus 1 according to the second embodiment as described above moves the PET detector rings 101 to outside the CT scanning range, triggered by an input of an instruction for CT scanning the subject P. That is, the PET-CT apparatus 1 of the second embodiment can move the PET detector rings 101 to the positions away from the CT scan irradiation range before X-ray irradiation due to a user selection of a CT scan, making it possible to reduce time-dependent degradation of the gamma-ray detectors 17 caused by scattered X-rays during CT scanning. Owing to such features, the PET-CT apparatus 1 of the present embodiment can elongate its longevity and achieve cost reduction as to maintenance and else.

To implement the technical ideas of some embodiments by an image generation/display method, the image generation/display method includes obtaining position information of each of the PET detector rings 101 in the axial direction of the bore 20, the PET detector rings 101 being movable relative to the table top 53 on which the subject P is to be laid in the axial direction of the bore 20 into which the table top 53 is to be inserted; generating the display information D1 representing positions of the PET detector rings 101, based on the position information; and displaying the display information D1 on the display 74. The procedures and effects of the image generation/display method are similar to or the same as those in the first embodiment, therefore, a description thereof is omitted.

To implement the technical ideas of some embodiments by an image generation/display program, the image generation/display program causes a computer to execute obtaining position information of each of the PET detector rings 101 in the axial direction of the bore 20, the PET detector rings 101 being movable relative to the table top 53 on which the subject P is to be laid in the axial direction of the bore 20 into which the table top 53 is to be inserted; generating the display information D1 representing positions of the PET detector rings 101, based on the position information; and displaying the display information D1 on the display 74. In this case the program for causing the computer to execute the image generation/display method can be stored in a storage medium such as a magnetic disk (e.g., hard disk), an optical disk (e.g., CD-ROM, DVD), or a semiconductor memory for distribution. The procedures and effects of the image generation/display program are similar to or the same as those in the embodiments, therefore, a description there of is omitted.

According to at least one of the embodiments described above, it is possible to move the positions of the PET detector rings 101 with improved efficiency.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A positron emission tomography (PET) apparatus, comprising:
a plurality of PET detector rings that is movable relative to a table top on which a subject is to be laid, in an axial direction of a bore into which the table top is to be inserted; and
processing circuitry configured to:
obtain position information of each of the plurality of PET detector rings in the axial direction,
generate display information representing positions of the plurality of PET detector rings, based on the position information, and
display the display information on a display.

2. The PET apparatus according to claim 1, wherein
the processing circuitry is further configured to:
obtain information as to the subject, and
generate the display information representing the positions of the plurality of PET detector rings and the information as to the subject in association with each other.

3. The PET apparatus according to claim 1, further comprising:
an input interface that receives an operation for moving one or more of ring objects on the display information, the ring objects individually representing the plurality of PET detector rings.

4. The PET apparatus according to claim 3, wherein
the processing circuitry is further configured to:
after setting positions of the ring objects, move one or more of the plurality of PET detector rings corresponding to the one or more of ring objects in the axial direction, based on the position information and the one or more of ring objects, and
update the display information along with the moving of the one or more of ring objects for display on the display.

5. The PET apparatus according to claim 1, further comprising:
an input interface that allows an input of a scanning range of the subject on the display information, wherein
the processing circuitry is further configured to:
select a scan mode close to the scanning range from a plurality of scan modes including a first mode and a second mode, the first mode being a mode in which the plurality of PET detector rings is uniformly arranged at predetermined intervals based on a body length of the subject, the second mode being a mode in which the plurality of PET detector rings is densely arranged for individual regions of the subject in the axial direction, and
display the selected scan mode and a possible scanning range according to the scan mode on the display together with the display information.

6. The PET apparatus according to claim 1, wherein
the processing circuitry is further configured to obtain, as the position information, projection positions of light projected vertically to the table top on which the subject is laid from a plurality of projectors individually included in the plurality of PET detector rings.

7. The PET apparatus according to claim 1, wherein
the processing circuitry is further configured to:
obtain coincidence event data based on output of a plurality of PET detectors individually included in the plurality of PET detector rings,
generate a count-rate map for each of the plurality of PET detector rings according to the coincidence event data, the count-rate map representing a gamma-ray count rate per unit time in each of the plurality of PET detectors, and
display the count-rate map on the display information displayed on the display such that the count-rate map is superimposed on each of the ring objects individually representing the plurality of PET detector rings.

8. The PET apparatus according to claim 1, wherein
the processing circuitry is further configured to:
in a first scan to be performed while the plurality of PET detector rings is uniformly arranged at predetermined intervals based on a body length of the subject, obtain coincidence event data based on output of a plurality of PET detectors individually included in the plurality of PET detector rings,
generate an accumulation map for each of the plurality of PET detector rings according to the coincidence event data, the accumulation map representing an accumulation distribution of gamma-ray counts, and
display a recommended arrangement of the plurality of PET detector rings for a second scan on the display information displayed on the display, the recommended arrangement being a dense arrangement of the plurality of PET detector rings about a position of one of the plurality of PET detector rings corresponding to an accumulation map with a least accumulation among the accumulation maps.

9. The PET apparatus according to claim 1, wherein
the processing circuitry is further configured to:
in a first scan of the subject, obtain coincidence events based on output of a plurality of PET detectors individually included in the plurality of PET detector rings,
generate a count-rate map for each of the plurality of PET detector rings according to the coincidence events, the count-rate map representing a gamma-ray count rate per unit time, and
display a switch object on the display information displayed on the display, triggered by an event that the gamma-ray count rate has reached a predetermined value, the switch object representing a switch from the first scan to a second scan.

10. The PET apparatus according to claim 1, wherein
the processing circuitry is further configured to:
in a first scan of the subject, move the plurality of PET detector rings in the axial direction such that the plurality of PET detector rings becomes uniformly arranged at predetermined intervals based on a body length of the subject,
after the first scan, move the plurality of PET detector rings or the table top on which the subject is laid in the axial direction such that the plurality of PET detector rings is individually placed at positions corresponding to the predetermined intervals, and
display, on the display, the display information such that a positional relationship between the plurality of PET detector rings and the table top differs during the first scan and during a second scan subsequent to the first scan.

11. A positron emission tomography (PET)-computed tomography (CT) apparatus, comprising:
a plurality of PET detector rings that is movable relative to a table top on which a subject is to be laid, in an axial direction of a bore into which the table top is to be inserted;
a CT imaging system configured to perform a CT scan of the subject;
an input interface that allows an input of an instruction for performing the CT scan; and
processing circuitry configured to move the plurality of PET detector rings away from an imaging range of the CT scan, triggered by the input of the instruction.

12. An image generation and display method, comprising:
obtaining position information of each of a plurality of PET detector rings in an axial direction of a bore into which a table top is to be inserted, the plurality of PET detector rings being movable in the axial direction relative to the table top on which a subject is to be laid;
generating display information representing positions of the plurality of PET detector rings, based on the position information; and
displaying the display information on a display.

13. A nonvolatile, computer-readable storage medium storing an image generation and display program for causing a computer to execute:
obtaining position information of each of a plurality of PET detector rings in an axial direction of a bore into which a table top is to be inserted, the plurality of PET detector rings being movable in the axial direction relative to the table top on which a subject is to be laid;
generating display information representing positions of the plurality of PET detector rings, based on the position information; and
displaying the display information on a display.
